(19) United States
(12) Patent Application Publication
(10) Pub. No.: US 2017/0055975 A1
(43) Pub. Date: Mar. 2, 2017

(54) ADJUSTABLE, LOCKING ALL-SUTURE ANCHOR ASSEMBLY AND METHOD FOR REPAIR

(71) Applicant: Raymond Thal, McLean, VA (US)

(72) Inventor: Raymond Thal, McLean, VA (US)

(21) Appl. No.: 15/251,744

(22) Filed: Aug. 30, 2016

Related U.S. Application Data
(60) Provisional application No. 62/213,274, filed on Sep. 2, 2015.

(51) Int. Cl. A61B 17/04

(52) U.S. Cl. CPC A61B 17/0401; A61B 2017/0408; A61B 2017/0427

(57) ABSTRACT

An adjustable, locking all-suture anchor assembly made up of a suture strand having a first end and a second end and an all-suture anchoring element. The suture strand and the all-suture anchoring element being entangled in a manner defining openings through which the first and second ends of the suture strand are passed such that upon bunching of the all-suture anchoring element and compression of the all-suture anchoring element is caused in a manner creating outward forces that allow for placement and anchoring of the all-suture anchoring element within a bone channel while simultaneously locking the first and second ends within the openings.

### BACKGROUND OF THE INVENTION

### 1. Field of the Invention

The present invention relates to devices or methods used in tissue repair, and more particularly to an adjustable, locking all-suture anchor assembly and a method for attachment of biological tissue (i.e., tendons or ligaments) to a bone mass.

### 2. Description of the Related Art

Soft tissues, such as tendons and ligaments, generally are attached to bone by small collagenous fibers. These connections are strong but permit the tendons and ligaments to be flexible. When a tissue, or a portion of a tissue, is torn away from the bone and requires repair, a surgeon is often required to repair the detached soft tissue with sutures, which are passed through bone tunnels and tied. A number of devices have been developed for securing a ligament or tendon to a bone mass. These devices can be used in place of bone tunneling techniques. These attachment devices are usually applied through extensive surgical incisions and, in some circumstances, by arthroscopic surgical techniques. The placement of bone tunnels for repair can be difficult and generally requires large open incisions. Through the advent of arthroscopic surgery, where the surgeon looks into a joint cavity with an arthroscope, there has been a trend to repair soft tissues back to bone through small incisions called portals.

A variety of devices are available for attaching objects to bone, such as screws, staples, suture anchors, and sutures alone. These devices have been used to attach soft tissue, such as ligaments, tendons, muscles to bone. A suture anchor assembly is a device, which utilizes small anchors, including those made of suture material alone, with additional suture materials attached thereto. A device, such as a screw, is inserted into the bone mass and anchored in place. After insertion of the anchor device, the attached suture is passed through the tissue to be repaired. The tying of a knot in the suture is then required to secure the tissue to the bone. The process of passing the anchored suture through the soft tissue and tying a knot is time consuming and difficult to undertake in the tight space encountered during arthroscopic surgery and sometimes even in conventional open surgery.

Knotless anchor assemblies have been popular and are embodied in a number of prior patents such as U.S. Pat. No. 5,709,708 wherein there is provided an assembly with an anchor means having a snag means and a loop suture element attached thereto. The suture loop is passed through the tissue to be repaired. The snag means then captures the loop suture element. The anchor is then inserted into a drill hole in a bone mass and the anchor locks into the bone. As the anchor is inserted into the drill hole, the tissue is pulled into secure attachment with a bone mass.

Further, in U.S. Pat. No. 6,045,574 there is provided an assembly with an anchor means having a snag means, and a hollow sleeve element with a loop suture element attached thereto. The snag means captures a loop suture element of the hollow sleeve element to draw tissue into secure attachment with a bone mass.

Further, there is provided an all-suture anchor assembly, such as disclosed in U.S. Patent Application Publication No. 2012/0290004 having an all fibrous construct, which is incorporated by reference. The device requires the tying of a knot to complete the surgical repair.

However, difficulties still exist with the all-suture anchor assembly and the present invention attempts to address these with a method and apparatus for adjustable, knotless anchoring using an all-suture anchor assembly.

### SUMMARY OF THE INVENTION

It is, therefore, an object of the present invention to provide an adjustable, locking all-suture anchor assembly that does not require the tying of a knot. The adjustable, locking all-suture anchor assembly is composed of an all-suture anchor assembly with an all-suture anchoring element to hold the two ends of the suture and enable passing through the all-suture anchoring element. The suture ends are threaded through the all-suture anchoring element for completion of a tissue repair. The all-suture assembly after threading of the suture ends through the all-suture anchoring element allows for the two ends to be pulled thereby drawing the tissue to the bone to effectuate a repair. The suture or sutures may thereby have their length for a repair adjustable in size.

It is also an object of the present invention to provide an adjustable, locking all-suture anchor assembly wherein the suture or sutures that are attached to or used with the anchor optionally have one, or more, barbs, beads, knots or capturable means attached thereto.

It is also an object of the present invention to provide an adjustable, locking all-suture anchor assembly including a hollow cylindrical mounting sleeve having an open distal end and an open proximal end defining a passageway therethrough. The assembly also includes a suture or sutures and an all-suture anchoring element provided as part of the all-suture anchor assembly, so that when the all-suture anchoring element is grabbed by the end of an inserter, both the all-suture anchoring element and sutures threaded therethrough are placed into the bone channel during a tissue to bone repair.

It is another object of the present invention to provide a method for securing tissue to bone which includes passing two ends of the suture through the all-suture anchoring element, and then enabling the suture and all-suture anchoring element to be captured with an end of an inserter. The all-suture anchoring element with the suture ends passed therethrough is inserted into a drilled bone channel or a mounting sleeve in a drilled bone channel. The suture or sutures that have been inserted into the bone channel or mounting sleeve along with the all-suture anchor in the bone channel are trapped when the all-suture anchor is deployed and the two ends of the suture are exposed to allow drawing and securing of the tissue to the bone. Optionally, the length of the suture or sutures can be adjusted, to effectuate a repair. Once inserted, the all-suture anchoring element of the all-suture anchor assembly holds the suture or sutures in the bone channel along with the all-suture anchoring element. Alternatively, the length of the suture or sutures can be adjusted by pulling on the ends suture or sutures.

It is another object of the present invention to provide a method for securing tissue to bone, wherein the assembly includes a cylindrical mounting sleeve which has an open proximal end and an open distal end defining a cylindrical passageway allowing access therethrough. The suture and all-suture anchoring element are then inserted into the sleeve contained in the bone channel.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 shows the adjustable, locking all-suture anchor assembly.

FIG. 2 shows the adjustable, locking all-suture anchor assembly where the first and second ends of the suture strand have been pulled through the tissue for subsequent engagement with the all-suture anchoring element of the all-suture anchor assembly by threading the first and second ends therethrough for placement into a bone channel.

FIG. 3 shows the adjustable, locking all-suture anchor assembly where the first and second ends of the suture strand have been pulled and threaded through the all-suture anchoring element of the all-suture anchor assembly for placement into a bone channel.

FIG. 4 shows the inserter end of the delivery inserter capturing the bunched all-suture anchoring element that has the suture strand threaded therethrough for placement into a bone channel.

FIG. 5 shows the inserter end of the delivery inserter that has captured the all-suture anchor assembly with the all-suture anchoring element and pushed the all-suture anchor assembly into a bone channel.

FIG. 6 shows a completed repair where the adjustable, locking all-suture anchor assembly has been placed into a drilled bone channel thereby drawing the tissue to a bone surface for a repair.

FIG. 7 shows a bone channel and a cylindrical mounting device that is inserted into the bone channel for an alternate embodiment of a repair.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as a basis for teaching one skilled in the art how to make and/or use the invention.

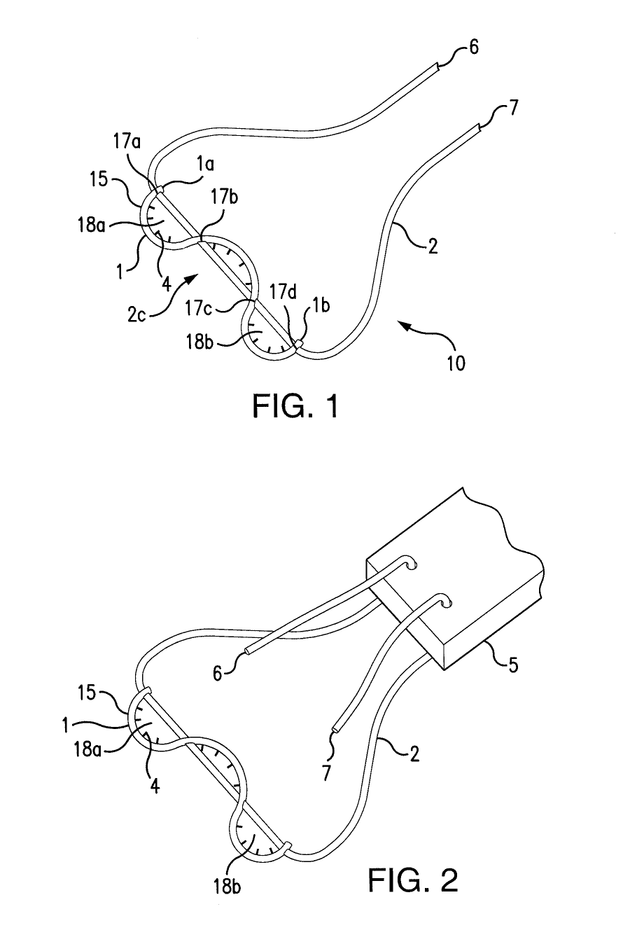

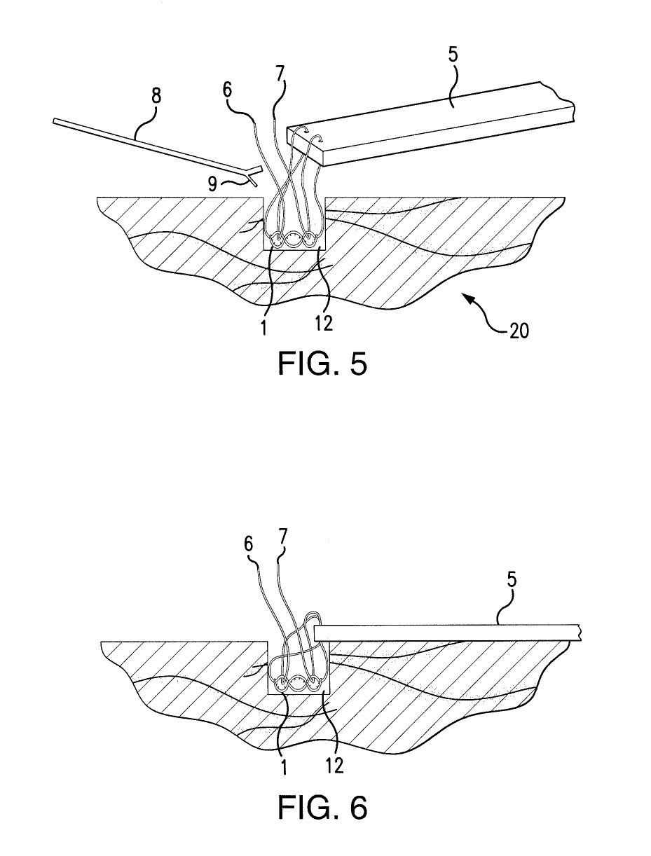

In accordance with the present invention, and with reference to FIGS. 1-7, an adjustable, locking all-suture anchor assembly 10 is disclosed. As will be appreciated based upon the following disclosure, the all-suture anchor assembly 10 includes an suture strand 2 and an all-suture anchoring element 1 (composed entirely of suture material as will be discussed below in greater detail and therefore may be referred to as an all-suture anchor), and a delivery inserter 8.

Considering first the all-suture anchor assembly 10, it is composed of a suture strand 2 and an all-suture anchoring element that are entangled in a manner facilitating bunching of the all-suture anchoring element 1, causing compression of the all-suture anchoring element 1 in a manner creating outward forces that allow for placement and anchoring of the all-suture anchoring element 1 within a bone channel 12. The all-suture anchoring element 1 is preferably composed solely of an enlarged piece of cylindrical suture material or a suture tape. The enlarged surface area of the all-suture anchoring element 1 allows for the passage of the suture strand 2 therethrough in a manner providing for entanglement of the all-suture anchoring element 1 and the suture strand 2. The all-suture anchoring element 1 includes a first end 1a and a second end 1b, as well as a thickness, a width and a length along a longitudinal axis.

As briefly mentioned above, the all-suture anchor assembly 10 includes the suture strand 2, which is passed through the all-suture anchoring element 1 at various locations 17a-d along the length of the all-suture anchoring element 1. That is, the suture strand 2 penetrates and traverses the all-suture anchoring element 1 so as to define apertures in the all-suture anchoring element 1. The intersections of the suture strand 2 with the all-suture anchoring element 1 are referred to herein as suture aperture locations and, as such, each of the suture aperture locations 17a-d is a location where the suture strand 2 passes through the thickness of the all-suture anchoring element 1. As shown in the figures, there are four suture aperture locations 17a-d. It is appreciated that as few as three suture aperture locations on a particular all-suture anchor assembly 10 may function well. Similarly, more suture aperture locations may be provided, although it has been discovered that each additional suture aperture location increases friction against the suture strand 2 thus reducing a surgeon's ability to slide the suture strand 2 in relation to the all-suture anchoring element 1. While the suture aperture locations 17a-d disclosed above in accordance with a preferred embodiment are all centrally located along the all-suture anchoring element 1 so as to be oriented along the central longitudinal axis of the all-suture anchoring element 1, it is appreciated the suture aperture locations may be varied (for example, staggered on opposite sides of the central longitudinal axis of the suture anchor) without departing from the spirit of the present invention. In accordance with a preferred embodiment, the all-suture anchoring element 1 may have various length and width dimensions depending upon the purpose for which it is intended.

With regard to the ultimate attachment of the first and second ends 6, 7 of the suture strand 2 to the all-suture anchor assembly 10 in accordance with the present invention, the first and second ends 6, 7 are preferably passed between the suture strand 2 and the all-suture anchoring element 1 at openings 18a, 18b positioned between the suture aperture locations 17a-d such that when the all-suture anchor assembly 10 is contracted to its compressed state, whether within a bone channel 12 or at another location within the body, friction is created between the first and second ends 6, 7 of the suture strand 2 and the all-suture anchor assembly 10 in a manner securing the first and second ends 6, 7 of the suture strand 2 to the all-suture anchor assembly 12. Alternately, the suture ends 6, 7 can be passed through any part of the all-suture anchor assembly 10, such as both suture ends can be both passed through either aperture 18a, 18b or through the openings 17a, 17b, 17c, 17d that have been created in the suture strand 2.

In accordance with a preferred embodiment, the all-suture anchoring element 1 is provided with barbs 4, or similar grasping feature, to hold the first and second ends 6, 7 of the suture strand 2 once it has been passed through the all-suture anchoring element 1 as described herein. In particular, once the first and second ends 6, 7 have been passed through the all-suture anchoring element 1 and the all-suture anchoring element 1 is contracted to its compressed state, the first and second ends 6, 7 are grasped by the barbs 4, which hold the first and second ends 6, 7 of the suture strand 2 in position along the length of the all-suture anchoring element 1. One particular type of barb 4 is a one-way type barb mechanism which grips the suture strand 2 and prevents the suture strand 2 from backing out of the entanglement of the all-suture anchoring element 1. While barbs 4 are disclosed in accordance with a preferred embodiment, it is appreciated embodiments without barbs may be used within the spirit of the present invention.

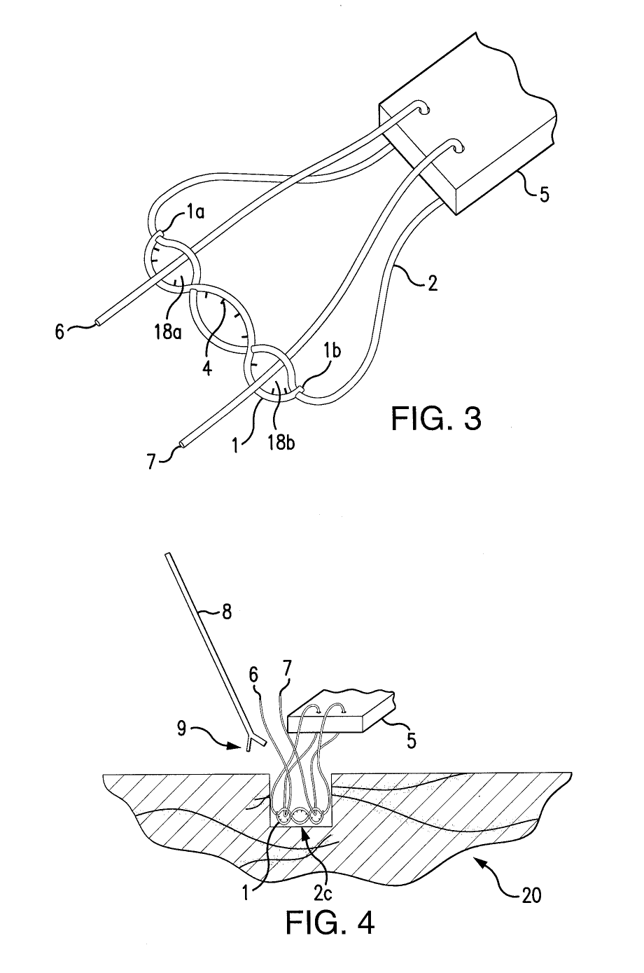

Attachment of the all-suture anchor assembly 10 to bone 12 is achieved in the following manner. Referring now to FIG. 3, and once the bone channel 12 is formed, the all-suture anchor assembly 10, in particular, the all-suture anchoring element 1 and the central portion 2c of the suture strand 2, are then inserted into the bone channel 12. It should be appreciated that the relative fit of the all-suture anchor assembly 10 in the bone channel 12 is shown as being relatively “loose.” This is done to provide for a clear view for the elements making up the present invention. In practice, it is appreciated that the suture strand 2, all-suture anchoring element 1, and delivery inserter 8 would be tightly pressed into the bone channel 12, as any excess space would need to be taken up by the expansion of the all-suture anchor assembly 10 in a direction substantially perpendicular to the longitudinal axis of the bone channel 12 (or otherwise oriented to contact side walls of the bone channel 12).

In particular, the delivery inserter 8 is operated by a surgeon whereby he captures the all-suture anchoring element 1 and the suture strand 2, which has its first end 6 and its second end 7 passed at least once through the all-suture anchoring element 1 and the suture strand 2 as described above. That is, the first and second ends 6, 7 are passed between the suture strand 2 and the all-suture anchoring element 1 at openings 18a, 18b positioned between the suture aperture locations 17a-d such that when the all-suture anchor assembly 10 is contracted to its compressed state, whether within a bone channel 12 or at another location within the body, friction is created between the first and second ends 6, 7 of the suture strand 2 and the all-suture anchor assembly 10 in a manner securing the first and second ends 6, 7 of the suture strand 2 to the all-suture anchor assembly 10. Alternately, the suture ends 6, 7 can be passed through any part of the all-suture anchor assembly 10, such as both suture ends can be both passed through either aperture 18a, 18b or through the openings 17a, 17b, 17c, 17d that have been created in the suture strand 2. The first and second ends 6, 7 of the suture strand 2 are gripped by the all-suture anchoring element 1 (or optionally by the barbs 4 located on the all-suture anchoring element 1). The surgeon inserts both the all-suture anchoring element 1, with the passed through now entangled first and second ends 6, 7 of the suture strand 2 into the bone channel 12 by pushing the inserter end 9 of the delivery inserter 8 into the bone channel 12. At that point, the surgeon can push a button or turn a device on the delivery inserter 8 which enables the deployment of the all-suture anchoring element 1 and then the surgeon removes the delivery inserter 8.

As shown in FIGS. 2 and 3, the suture strand 2 and the all-suture anchoring element 1 are installed in an elongated fashion to take a small diameter configuration, referred to herein as an undeployed state or installation state. Deployment occurs as the central portion 2c of the suture strand 2 and the all-suture anchoring element 1 are positioned in the bone channel 12 and the suture strand 2 is tensioned causing the all-suture anchoring element 1 to fold, bend, crease, crinkle, bunch or otherwise change shape in a manner that compresses the all-suture anchoring element 1. As the all-suture anchoring element 1 is compressed in this manner it ultimately increases in size in a direction substantially perpendicular to the longitudinal axis of the bone channel 12 (or otherwise oriented to contact side walls of the bone channel 12) and develops an outwardly directed forced that is directed at the walls of the bone channel 12. As such, and when tension is no longer being applied by the delivery inserter 8, or other delivery instrument, the all-suture anchoring element 1 exhibits an outward bias resulting in expansion of the all-suture anchoring element 1 in a direction substantially perpendicular to the longitudinal axis of the bone channel 12 (or otherwise oriented to contact side walls of the bone channel 12) into which it is positioned, resulting in frictional engagement or gripping the wall of the cancellous bone, which is referred to herein as the expanded deployed state.

As shown, the all-suture anchoring element 1 is folded or otherwise compressed to form pleats between adjacent suture aperture locations 17a-d. This pleating reduces the distance between the first suture aperture location 17a and the second suture aperture location 17b, as measured along the length of the all-suture anchoring element 1. These pleats form a bunched mass of suture material effectively increasing a diameter, or cross sectional dimension, (as measure in relation to the axis of the bone channel 12) of the all-suture anchoring element 1, which ultimately causes the all-suture anchor assembly 10 to displace cancellous bone. The relative increase in the cross sectional size of the all-suture anchoring element 1 in the direction substantially perpendicular to the longitudinal axis of the bone channel 12 (or in another direction to facilitate contact of the all-suture anchoring element 1 with the side walls of the bone channel 12) creates a retention force of the all-suture anchor assembly 10.

It is appreciated that increasing the number of suture aperture locations 17a-d from the four shown to five, six, seven or more is likely to increase the size of the all-suture anchor assembly 10 after deployment and the number of pleats is therefore increased. However, a limiting factor is the amount of friction increased by additional suture aperture location.

With the foregoing in mind, it is appreciated that a large variety of constructions and materials will work for the all-suture anchor assembly 10. It has been discovered that for each type of construction (i.e., braided, woven, non-woven, or knitted) there is an advantage for using a material that increases in width for every reduction in length. This advantage provided for increased diameters for a particular number of folds, pleats, crinkles or other changes in the shape of the all-suture anchoring element 1. Regardless of the material chosen for use in accordance with the present invention, the material must exhibit desirable deformation and retention characteristics.

It is appreciated a mechanical tensioning mechanism, as is well known to those skilled in the art, may be used during the deployment of the all-suture anchoring element 1. Such mechanical tensioning mechanisms pull or ratchet the suture strand 2 while the delivery inserter 8 holds the all-suture anchoring element 1 in place. Mechanical tensioning, of this nature, may be preferable as this can more tightly ‘fold’ or ‘bunch’ the all-all-suture anchoring element 1, thereby increasing the created tension, that is, the outward force of the all-suture anchoring element 1, which is relative to the longitudinal axis of the bone channel 12 and toward the walls of the bone channel 12, resulting from increased compression of the all-suture anchoring element 1. The changed shape of the all-suture anchoring element 1 provides security within the bone, for example, below the cortical layer.

Prior to expansion and folding (or otherwise changing the shape) of the anchor suture 1, the first and second ends 6, 7, as well as the remainder of the suture strand 2, are tensioned as desired to hold tissue (or other bodily material) to be retained close to the bone 20. With the sutures tensioned, the suture strand 2 is tensioned (either by manually or mechanically pulling this suture strand 2) to cause the all-suture anchoring element 1 to fold and expand as explained above. In addition to causing the all-suture anchoring element 1 to grip the wall of the cancellous bone channel 12, the tensioning of the suture strand 2 and the resulting folding of the all-suture anchoring element 1 causes the frictional engagement between the central portion 2c of the suture strand 2, the first and second ends 6, 7 of the suture strand 2, and the all-suture anchoring element 1. In particular, and as the all-suture anchoring element 1 is compressed during the deployment process, the all-suture anchoring element 1 and the suture strand 2 are brought closer together in a tightly compacted and entangled configuration that effectively creates a unitary mass that ultimately prevents the central portion 2c of the suture strand 2 from being pulled away from the all-suture anchoring element 1 and also prevents the first and second ends 6, 7 of the suture strand 2 from being pulled away from the central portion 2c of the suture strand 2 and the all-suture anchoring element 1. It is appreciated that bunching and expansion of the all-suture anchoring element 1 may be assisted by the use of a button or device. It is also appreciated that any all-suture anchoring element 1 which deploys or bunches when inserted into a bone channel 12 could be usable along the suture strand 2, especially those which have at least one barb 4 or gripping feature device placed on the all-suture anchoring element 1.

As briefly mentioned above, the present invention achieves secure attachment of soft tissue 5 to a bone mass 20 using the present adjustable, locking all-suture anchor assembly repair assembly 10. The suture strand 2 once threaded through the tissue 5, at least once, has its first end 6 and second end 7 passed through an all-suture anchoring element 1 located along the suture strand 2 to allow the ends 6, 7 of the suture strand 2 to be threaded therethrough (see FIG. 3). As explained above, the all-suture anchoring element 1 optionally has a barb 4 or gripping feature device to prevent the first and second ends 6, 7 of the suture strand 2 from backing out of the all-suture anchoring element 1. It is also appreciated the suture strand 2 may be adjustable in length by adding a slip knot 14 along the length of the suture strand 2. It is also appreciated that once the all-suture anchoring element 1 and suture strand 2 with the first and second ends 6, 7 threaded therethrough is inserted into the bone channel 12 by the inserter end 9 of the delivery inserter 8, a surgeon can draw of the tissue 5 to the bone 20 by pulling of the first and second ends 6, 7 of the suture strand 2 after the all-suture anchoring element 1 has been deployed or bunched.

In accordance with the present method, the first and second ends 6, 7 or the suture strand 2 are first passed, at least once, through the soft tissue 5, which one wishes to secure to the bone mass 20 (see FIG. 2). Referring to FIGS. 3-6, the first and second ends 6, 7 or the suture strand 2 are then threaded through the all-suture anchoring element 1 and frictionally retained therein or grabbed by the barbs 4 or gripping features located on the all-suture anchoring element 1. Once pulled through the soft tissue 5, the first and second ends 6, 7 or the suture strand 2 are fed, at least once, through openings created by the combination of the central portion 2c of the suture strand 2 and the all-suture anchoring element, which together form the all-suture anchoring assembly 10. As discussed above, if suture strand 2 is relatively long, it may be constructed so as to allow for adjustment in length. Once the first and second ends 6, 7 or the suture strand 2 have been threaded through the openings 18a, 18b created by the all-suture anchoring element 1 and the central portion 2c of the suture strand 2, and gripped by the all-suture anchoring assembly 10 and/or the barbs 4 on the all-suture anchoring element 1, the all-suture anchoring element 1 is inserted by the inserter end 9 of the delivery inserter 8 into the bone channel 12, causing deployment or expansion of the all-suture anchoring element 1 in the bone channel 12. The tissue 5 may then be drawn to the bone 20 by pulling the first end 6 and the second end 7 of the suture strand 2.

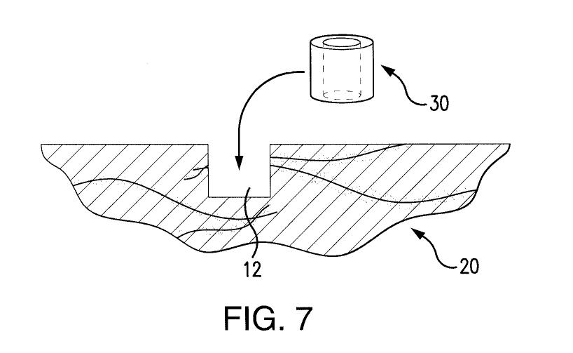

Referring to FIG. 7, it is also appreciated that a sleeve 100 (likely threaded) might be inserted into the bone channel 12 and the all-all-suture anchoring element 2 is then placed into the sleeve 100. One goal of such a sleeve 100 would be to enhance fixation of the all-suture anchoring element 2. In accordance with such an embodiment, the all-suture anchoring element 2 would achieve fixation by gripping the walls of the sleeve 100. While a screw-in method of insertion is contemplated with the threaded construction, the sleeve may also be pressed into position or deployed in some other manner. The sleeve need not be uniform, so long as it can be secured within the bone channel, likely along the perimeter thereof. Alternatively, the all-suture anchoring element can change shape ‘distal’ to the sleeve thereby providing fixation. The sleeve would be positioned in the cortical bone aspect of the bone channel, but could also extend into the cancellous bone. It is, however, appreciated the sleeve could be slightly proud and not flush with the cortical surface, or it could be placed below the cortical surface, as long as it is well-fixed. It is also appreciated that when the all-suture anchoring element is deployed distal to the sleeve, that is, between the sleeve and the bottom of the bone channel, fixation doesn't require ‘force’. Rather, the enlargement and increased size (in a direction toward the walls of the bone channel) of the all-suture anchor upon deployment, distal to the sleeve after insertion, prevents the all-suture anchoring element from backing out of the sleeve (and ultimately from backing out of the bone channel). The mounting sleeve 100 is variable in size and defines a hollow cylindrical body with a central passageway. As such, the mounting sleeve 100 includes a distal end and a proximal end. The mounting sleeve 100 is open, or hollow, as it extends from the distal end thereof to the proximal end thereof allowing access below the mounting sleeve 100 to the internal bone mass 20. The mounting sleeve 100 is in the form of an open cylindrical mounting sleeve with or without external threading. It is appreciated any fixation method can be utilized to affix the mounting sleeve 100 to the bone mass 20. It is appreciated the hollow cylindrical mounting sleeve 100 provides an anchor recess (or central passageway) extending from the proximal end of the hollow cylindrical mounting sleeve 100 to the distal end of the hollow cylindrical mounting sleeve 100. The central passageway, in the hollow cylindrical mounting sleeve 100, allows for capture of the combination of the all-suture anchoring element 1, optionally with the barbs 4 thereon, along with the suture 2, when they are both forced into the central passageway by an inserter end 9. The all-all-suture anchoring element 1 will be deployed upon removal of the inserter 8 and will expand outward to create compression or a friction fit with the bone channel 12 or within the hollow cylindrical mounting sleeve 100. Alternatively, the all-suture anchor assembly 10 will pass through the mounting sleeve 100 and deploy and lock below the distal end of the mounting sleeve 100. An alternative, more extensive, repair can comprise multiple drilled bone channels. When multiple drilled bone channels are used, multiple adjustable, locking all-suture anchor assembly repair assemblies are used. Each assembly is used as described above and inserted into a separate drilled bone channel or a mounting sleeve placed in the drilled bone channel. This enables a surgeon to grab various sections of a tissue and draw the tissue to bone at several locations to secure the tissue for a repair.

In many situations throughout the discussion above, the terminology relating to the secure attachment of soft tissue to bone mass has been used. Such terminology refers to the attachment or reattachment of tissue to a bone mass by securely binding the tissue to the bone mass utilizing the novel knotless suture anchor assembly. The suture element can be made up of a known suture material, or it can be made of polymer materials, or can be formed of bioabsorbable/biocomposite material such as a polylactide polymer.

While the preferred embodiments have been shown and described, it will be understood that there is no intent to limit the invention by such disclosure, but rather, is intended to cover all modifications and alternate constructions falling within the spirit and scope of the invention.

What is claimed is:

1. An adjustable, locking all-suture anchor assembly, comprising:
a suture strand having a first end and a second end;
an all-suture anchoring element;
the suture strand and the all-suture anchoring element being entangled in a manner defining openings through which the first and second ends of the suture strand are passed such that upon bunching of the all-suture anchoring element, compression of the all-suture anchoring element is caused in a manner creating outward forces that allow for placement and anchoring of the all-suture anchoring element within a bone channel while simultaneously locking the first and second ends within the openings.

2. The all-suture anchor assembly according to claim 1, wherein the all-suture anchoring element is composed solely of an enlarged piece of cylindrical suture material or a suture tape.

3. The all-suture anchor assembly according to claim 1, wherein the suture strand is passed through the all-suture anchoring element at various locations along a length of the all-suture anchoring element so as to define the openings between the positions at which the suture strand is passed through the all-suture anchoring element.

4. The all-suture anchor assembly according to claim 3, wherein the suture strand penetrates and traverses the all-suture anchoring element so as to define apertures in the all-suture anchoring element.

5. The all-suture anchor assembly according to claim 1, wherein the all-suture anchoring element includes barbs shaped and dimensioned to hold first and second ends of the suture strand once it has been passed through the openings.

6. The all-suture anchor assembly according to claim 1, further including a sleeve shaped and dimensioned for insertion into a bone channel so as to receive the all-suture anchoring element and enhanced fixation thereof.
